(19) United States
(12) Patent Application Publication
(10) Pub. No.: US 2004/0001847 A1
(43) Pub. Date: Jan. 1, 2004

(54) Method and composition to elicit an effective autologous antitumoral immune response in a patient

(76) Inventor: Eduardo M. Lasalvia-Prisco, Hollywood, FL (US)

Correspondence Address: MALLOY & MALLOY, P.A., Historic Coral Way, 2800 S.W. Third Avenue, Miami, FL, 33129, US

(21) Appl. No.: 10/607,358

(22) Filed: Jun. 26, 2003

Related U.S. Application Data
(60) Provisional application No. 60/391,674, filed on Jun. 26, 2002.

(51) Int. Cl. A61K 39/00

(52) U.S. Cl. 424/185.1

(57) ABSTRACT

A method and a composition to elicit an effective antitumoral immune response in a patient, specific to his or her own tumor antigens (i.e. an autologous antitumoral immune response), wherein the method preferably elicits an effective autologous antitumoral immune response in a cancer patient. The method includes generating, preserving, and storing specific tumor associated antigens or TAA, and eliciting the autologous antitumoral immune response, at least in part, through a combination of dual vaccines. Specifically, the method provides for both an internal and external vaccine. Additionally, the preparation of an autologous hemoderivative composition for utilization as the an external vaccine is presented, as well as the autologous hemoderivative composition itself.

### CLAIM OF PRIORITY

The present application is based on and a claim to priority is made under 35 U.S.C. Section 119(e) to the provisional patent application currently pending in the U.S. Patent and Trademark Office having Serial No. 60/391,674 and a filing date of Jun. 26, 2002.

### BACKGROUND OF THE INVENTION

1. Field of the Invention

The present invention relates in general to a method and a composition to elicit an effective antitumoral immune response in a patient, specific to his or her own tumor antigens (i.e. an autologous antitumoral immune response). More specifically, the present invention relates to a method to elicit an effective autologous antitumoral immune response in a cancer patient which comprises generating, preserving, and storing specific tumor associated antigens, and eliciting the autologous antitumoral immune response, at least in part, through a combination of dual vaccines. The present invention further provides for enhancement of the antitumoral immune response resulting from an internal vaccine and an external vaccine by activating antigen presenting cells, as well as by inhibiting a tolerance immune response in cancer patients. The present invention further provides a method for preparing an autologous hemoderivative composition for utilization in the inventive method as an external vaccine.

2. Description of the Related Art

Initially, immunotherapy techniques began with treatments or vaccines for the prevention of infectious diseases, and they have since virtually eradicated many such diseases. Generally, immunotherapy has exploited the ability of an immune system to respond when it is in contact with alien molecules known as antigens. An immune response is specifically addressed against antigenic molecules or against other organisms that express these antigenic properties, collectively known as antigens. When the antigen is not a living organism, the immune response is frequently mediated by cells identified as helper cells or CD4+ lymphocytes, and by antibody producing cells, the final effectors being antibodies, for example, immunoglobulin molecules that are circulating throughout a patient's bloodstream. When the antigen is a component of a live cell or microorganism, the immune response is mediated by circulating cells and also the effector cells, mainly, cytotoxic CD8+ lymphocytes.

Frequently, the presence of antibodies against a specific antigen can be tested by the immediate (20-60minute) response obtained when a dermic test with the antigen is performed. The cellular response is tested by a delay (48-72 hour) response after dermic exposure to the antigen. As a consequence, the antibodies and the cellular immune response to them are also known as immediate response or delay response, respectively.

Most of the time, the immune response is associated with cooperation between the antibodies, or the molecular mediated arm, and the cellular mediated arm. Typically, the cooperative response is the antibodies known dependent cytotoxic response. Classical immunotherapy techniques have used such antigens as vaccine agents. These agents were treated to avoid their pathogenicity and/or they were mixed with adjuvants in order to facilitate their accessibility, recognizance or stimulant activity. Antigens are necessary for immune response because, by definition, an immune response is a specific antigen-addressed response, however, modern research has recognized that sometimes although antigens are present, their immunological power is not enough to stimulate an effective immune response. In such cases, the immune response can be elicited by other substances or by modified antigens with more powerful antigenic activity and cross-reactivity with the specific target of the immune response. In addition, some agents have been identified which elicit immune responses not upon specific antigens, but, rather, upon specific or global reactive portions of the immune system. As a consequence, today it is more appropriate to identify this whole family of compounds which may be used in immunotherapy, including specific antigens and all other agents that elicit or enhance a response against antigens or from the immune system, collectively, as immunogens.

It is appreciated that human cancer immunotherapy has been in use and has been subject of reported research for years. More in particular, human cancer immunotherapy began when specific antigens in malignant cells were recognized. With this knowledge, the stimulation of a patient's immune response against the specific antigens of these malignant cells as an antitumoral treatment was explored. Along with surgery, chemotherapy, and radiotherapy, immunotherapy provides yet another therapeutic technique available in Oncology. Frequently, these therapeutic techniques are employed simultaneously or successively in various treatment regimens.

Cancer immunotherapy techniques are commonly grouped into one of two categories, namely, non-specific immunotherapy or specific immunotherapy. The goal of non-specific cancer immunotherapy is an increase in all of a patient's immune responses, thereby improving the activity level throughout the patient's immune system. Specific cancer immunotherapy, on the other hand, has the goal of stimulating a singular antitumoral immune response that may be directed against the patient's tumor or the patient's tumor type or an antigen of that tumor.

Each of these immunotherapy techniques may be further grouped into sub-categories, being either an active immunotherapy or an adoptive immunotherapy. Active immunotherapy techniques comprise methods wherein the immune response induced by treatment is dictated by the patient's own immune system, whereas, adoptive immunotherapy techniques comprise methods in which one, several, or all of the components of the patient's immune system are replaced, thereby dictating an alternate immune response.

Thus, there are in actuality, four commonly known immunotherapy techniques utilized in the field of cancer treatment, specifically: active non-specific immunotherapy; adoptive non-specific immunotherapy; active specific immunotherapy; and, adoptive specific immunotherapy. Numerous agents have been produced, modified, or protocolized by different methods in order to be employed as immunogens in one or more of these four cancer immunotherapy techniques. Each these techniques, however, exhibit certain shortcomings which hinder their development and limit their implementation as effective and safe cancer treatment regimens, except for short periods of time and only for a small number of select tumor types. A description of each of these four cancer immunotherapy techniques is presented below in further detail, including the known shortcomings of each.

Active non-specific immunotherapy includes the administration of a biological or chemical agent that has been proven to stimulate immune system activity. Compositions comprising bacille Calmette-Guerin(BCG), Corynebacterium, levamisole, and zinc compounds have been among the most tested immunogens. The basic supposition is that cancer patients are always immune-depressive and that this technique could restore the immune system activity, including antitumoral response. It must be noted, however, that the supposed global depression of the immune system is yet to be demonstrated in most cancer patients, thus, the global immune-restoration is not necessarily a proper goal of treatment, and the possible secondary and potentially negative effects of non-specific immunotherapy are, therefore, not justified. In fact, in some cases it has been reported that non-specific hyper-stimulation of the immune system has also produced enhancement or tumor progression. This result may be explained by the stimulation of cell populations with properties of tolerance or suppression.

Adoptive non-specific immunotherapy comprises one variation involving the transfer of immunocompetent cell precursors, known as source cells, from a donor to a receptor in order to allow their proliferation, thereby resulting in the quimeric regeneration of immune system cell populations. In particular, tests have been performed on the transfer of a defined sub-population of immunocompetent cells to determine if it may increase their function. A common and well known treatment regimen utilizing this technique is an allogenic bone marrow transplant. One of the main drawbacks of this technique is the prevalence of reactions (i.e. rejection) of the transplanted bone marrow by the host.

More recently, another variation of adoptive non-specific immunotherapy has been tested which employs the administration of select components of the immune system which are candidates to promote a more amplified immune response. For example, recombinant molecules that are normally mediators of immune response, such as interferons and interleukins, have been the agents produced by genetic recombination and employed as immunogens in order to expand antitumoral responses. These compounds are known as biological response modifiers and they are active as antitumoral agents but only in a few specific types of tumors such as, renal cell carcinoma, melanoma, hairy cell leukemia, and non-Hodgkin's lymphoma. Additionally, even when used to treat these specific types of tumors, most of the benefits are partial and temporary. The problem with this technique appears to be that the rate-limiting step of antitumoral immune response and the target step of a biological response modifier acting in isolation are presently unknown. As a consequence, the effectiveness of any treatment with these agents is fortuitous, at best.

Adoptive specific immunotherapy is a method of treatment that uses lymphocytes which have previously been in contact with tumor cell antigens, either in vivo or in vitro. In addition, this immunotherapy technique uses recombinant monoclonal antibodies against specific molecular targets expressed by malignant tumor cells. The antecedents of this procedure are the treatment of infectious diseases with hyperimmune serum or immunoglobulins. Subsequently, in the field of cellular mediated immunity, the intent was to collect tumor infiltrating lymphocytes or dendritic cells and to re-inject them with previously known pulse activation or clonal expansion.

Currently, there is active development directed towards the use of recombinant monoclonal antibodies directed against molecular tumor targets which represents a variation of adoptive specific immunotherapy. Components of tumor receptors such as H2-neu and CD20, which may be over-expressed in cells of some breast cancers and non-Hodgkin's lymphomas, respectively, are the most effective target for the monoclonal antibodies currently available for immunotherapy. The effectiveness of the treatment of patients having malignant diseases with monoclonal antibodies also appears to be more the exception than the rule. The remissions are frequently limited to only a fraction of patients treated and having tumors with the supposed antigenic target, and these remissions are generally only temporary.

The difficulties encountered with this. immunotherapy technique appear to be that molecular changes or losses in the target of the transferred immune effector are very frequent due to malignant disease evolution. This is due, at least in part, because tumor cells exhibit a high rate of spontaneous mutation as a consequence of their high proliferative turnover and their high rate of mutation induced by oncological therapies. This results in an immunological escape mechanism which detracts from the effectiveness of this immunotherapy technique.

Lastly, active specific immunotherapy utilizes a vaccine comprising tumor specific antigens, known as neo-antigens or tumor associated antigens (TAA). The existence of TAA has been well recognized for some time. This immunotherapy technique includes all vaccine treatments that include the administration of antigens as tumor cells, tumor extracts, or as purified molecular compounds extracted from tumors. In order to enhance the elicited immune response, different procedures have been tested including alternate methods of inoculation (e.g. intradermal, subcutaneous, intramuscular, or intravenous), as well as the use of different adjuvants (e.g. tumor cells with genetic engineering to secrete immune-modulating cytokines, antigen pulsed dendritic cells, mixed antigens with BCG, tumor peptide antigens combined with chaperone heat shock proteins, and hapten potentiation of antigens).

In the last decade, active specific immunotherapy techniques have been developed utilizing autologous systems, the goal being to obtain a more specific immune response against well-demonstrated tumor cell antigens specifically expressed by an individual tumor. This represents a significant advance in active specific immunotherapy because it allows customization of the immunotherapy to an individual antigen profile that is generated in a specific tumor by spontaneous and therapeutically associated gene tumor cell mutations, through the individual patient-tumor history. Clinical assessment of such active specific immunotherapy techniques, however, indicates that it has only produced effective results in the treatment of a few tumors and, once again, the results obtained are only partial and are only temporary.

The difficulties encountered in active specific immunotherapy techniques are mainly twofold. The first problem is related to the basic nature of cancer itself. More in particular, the malignant cells derived from a normal patient are the patient's self-cells and, therefore, their molecular composition is not normally antigenic relative to the host (i.e. the patient's) immune system. The molecules in tumor cells that are unrecognizable as the patient's self-cells are products of etiological or therapeutical mutations and/or specific epigenetic structural modifications. The concentration of these antigenic compounds are typically low in most malignant tissues, and their antigenicity is further reduced because the antigens are normally stored within the malignant cells, far from the afferent immune system. Additionally, these stored antigens are frequently destroyed by proteolysis when the malignant tumor cells die by programmed death or apoptosis, unless they are first protected, such as by protein induced cell stress.

The second difficulty encountered in active specific immunotherapy relates to the preparation of a vaccine having the patient's malignant tumor as its source. Here, both quantitative and qualitative limitations are present. To being with, the number of inoculations and the amount of immunogen, or vaccine, in each inoculation as required by this technique are limited by the availability of surgical tumor specimens, and the typically weak antigens which are present at low cellular concentrations therein. In addition, and as noted above with respect to adoptive specific immunotherapy techniques, if tumor cells modify their antigenic profile due to their high rate of mutation, the immune effectors elicited by inoculation of the original vaccine may not recognize a target in the remaining mutated tumor cells. As a result, repeated inoculations of the original vaccine will not usually be effective unless current surgical tumor specimens are available in order to prepare vaccines containing the successively mutated antigens, however, such current surgical tumor specimens are hardly, if ever, available.

Thus, it would be beneficial to provide a method to elicit an antitumoral immune response in a cancer patient and, more in particular, an effective autologous antitumoral immune response thereby providing a new, improved, and innovative active specific immunotherapy technique. Additionally, it would be helpful to provide a method to elicit such an effective autologous antitumoral immune response in a cancer patient via a treatment regimen structured to modify an antigen library of tumor cells, or TAA, thereby increasing the antigenicity relative to the patient's immune system. It would also be desirable for such an improved method to utilize a dual vaccine regimen including both an internal vaccine comprising the endogenous release of TAA from the tumor itself, as well as an external vaccine comprising a composition derived from an autologous blood specimen obtained from the patient at a plurality of discreet time periods over the course of the entire treatment regimen. Any such method would further benefit from the provision of a procedure to enhance the antitumoral immune response in a patient via the activation of an antigen presenting cell or APC population, and to inhibit a tolerance immune response in the patient. In addition, a method for preparing a hemoderivative composition from an autologous blood specimen for use as an external vaccine would be desirable.

### SUMMARY OF THE INVENTION

In view of the many drawbacks inherent in the immunotherapy techniques currently known and used in the treatment of cancer, and as otherwise identified in the art, the present invention provides a method and a composition to elicit an antitumoral immune response in patients, thereby providing a new improved active specific technique for practicing cancer immunotherapy. It is noted that the method of the present invention comprises certain aspects of procedures known in medical practice and/or in medical research, such as treatment with cytokines, colony stimulating factors frequently used for hematological and immunological restoration, application of chemotherapy and indomethacin, which have been used and are the subject of continued research as antitumoral treatments, and the malignant cell autoschizis promoted by high-doses of ascorbic acid and menadione. Numerous other procedures employed by the present invention, however, are not known in the art, such as, by way of example only, an enhanced generation and subsequent in vivo storage of specific TAA in the tumor cells of a cancer patient, and a dual vaccine regimen including an internal vaccine comprising a release of previously generated, preserved, and stored endogenous TAA from the tumor cells of the patient, and an external vaccine comprising an autologous hemoderivative composition. The procedures utilized by the method of the present invention, whether previously known or initially presented herein, utilize various compounds which are known in human pharmacology and approved for medical practice all over the world. The innovation of the present invention lies in the way these known compounds and procedures are utilized in combination with the inventive procedures presented herein to achieve the objectives of the present invention.

It is hereby asserted that the present invention defines an inventive method which allows the practice of a new, improved, and innovative autologous active specific immunotherapy technique. More importantly, the method of autologous active specific immunotherapy defined by the present invention is distinguishable from all previously known immunotherapy techniques, such as those described above. As an initial matter, it is noted that the method of the present invention provides for an enhancement of tumor antigenicity relative to an immune system of a cancer patient as a distinguishing factor in autologous active specific immunotherapy. Another distinguishing factor is that the inventive method comprises a dual vaccine, one being an internal vaccine and another being an external vaccine. As previously indicated, one vaccine is an internal vaccine because after the storage of one or more immunogens in the patient's tumor cells, the patient is “vaccinated” by triggering the subsequent release of the immunogens (e.g. antigens, TAA, or vaccine) from the tumor cells to the interstitial spaces in the patient's body such as phagocytes, lymphatic vessels and/or blood vessels. The other vaccine is an external vaccine because the patient is vaccinated via a subcutaneous inoculation, or other inoculation technique, with a hemoderivative composition prepared from an autologous blood specimen containing the one or more immunogens (e.g. antigen, TAA, or vaccine).

As stated above, known autologous active specific immunotherapy techniques for cancer utilize a surgical specimen of a patient's tumor as a source of vaccine, which is another important distinction of the present invention. In particular, the known immunotherapy techniques which require surgical specimens of the patient's tumor inherently comprise as a limiting condition the availability of such a surgical specimen, which is rarely available more than once, and even then, it is more than likely during the initial stages of diagnosis and treatment. Therefore, it is not possible to update the vaccine when utilizing known immunotherapy techniques, as may be required if the remaining tumor changes its antigenic expression, which is not an uncommon occurrence given the high rate of mutation in such organisms.

Conversely, the present invention as described herein eliminates the need for surgical specimens of the patient's tumor, and rather utilizes the remnant tumor cells, and, more specifically, the neo-antigens and/or TAA released from the remnant tumor cells into the patient's bloodstream which provide the source of immunogens for both an initial internal vaccine, as well as for subsequent internal and external vaccines. As a result, the present invention provides for a plurality of vaccines which may be repeatedly updated so as to be effective against the specific tumor antigens as they change. This is possible because the present invention utilizes the antigen library of the patient's remaining tumor to provide the immunogen which is the target of the immune response elicited from the internal vaccine and is subsequently the source of the antigenic immunogen in the blood utilized to produce an external vaccine. As such, the immunogen, and thus, the internal and external vaccines, are always contemporary each time the inventive method is employed.

As may be seen from the foregoing, the present invention comprises a new, improved, and inventive method of autologous active specific immunotherapy which, while incorporating certain aspects of known cancer immunotherapy techniques, comprises numerous novel features which eliminate many of the shortcomings of these previously known techniques. Furthermore, none of the novel features of the method of the present invention are anticipated, rendered obvious, suggested, or even implied by any known immunotherapy technique or other cancer treatment described herein or otherwise known.

Turning now to a further description of the method of the present invention, it is generally directed towards eliciting an effective autologous antitumoral immune response in a cancer patient and comprises generating a plurality of neo-antigens or tumor associated antigens (TAA) in a plurality of tumor cells of the patient, preserving the plurality of TAA in the plurality of tumor cells, activating a plurality of antigen presenting cells (APC), breaking or inhibiting an immune tolerance response, triggering an internal vaccine in the patient, and providing the patient an external vaccine comprising an autologous hemoderivative composition. Additionally, the present invention comprises a method for the preparation of an autologous hemoderivative composition such as may be utilized in the foregoing method for eliciting an effective autologous antitumoral immune response, as well as the autologous hemoderivative composition. Additionally, the present invention comprises a method for performing an immunological assessment of an elicited immune response, as well as for performing a clinical assessment of an elicited antitumoral response.

To accomplish the objectives of the present invention, the method includes generating a plurality of neo-antigens or tumor associated antigens (TAA) in a plurality of tumor cells of the patient. The plurality of TAA may include peptides and/or proteins with molecular sites unrecognized as molecular components of the patient's self-cells and, therefore, of the normal organic composition, by the patient's immune system. These alien molecules (i.e. TAA) are generated in malignant tumors, by genome abnormalities or mutations. The mutated genes can be attributed to etiopathogenesis of cancer (oncogenes, antioncogenes), physiopathology of cancer (high proliferation fraction with high rate of spontaneous mutations), or therapeutical interventions (radio- and chemo-induced mutations). Mutated genes can generate a plurality of TAA by their direct expression or by the promotion of intracellular conditions eliciting epigenetic normal protein transformation. In order to generate a plurality of TAA in tumor cells, it is necessary to increase in these cells their protein synthesis and mutation frequency.

Thus, the method also comprises inducing protein synthesis in a plurality of tumor cells by treating the patient with a suitable pharmaceutical compound in order to activate the growth factor-receptors, such as are typically highly expressed in most malignant cells. One pharmaceutical compound which is suitable for this purpose is insulin, due to the insulin-like growth factor-receptors which are highly expressed in many malignant cells.

Insulin action requires the agonism of a cellular insulin-receptor. As result of this agonism, the receptor is activated and several biological processes are started. Among the processes activated by insulin-receptor agonism are protein synthesis linked to the incorporation from outside the cell of amino acids. In addition, the known insulin-like growth factors (1 and 2) have their cell receptors, and their agonism promotes the tumor cell growth and, therefore, the tumor cell protein synthesis. Insulin-like growth factors are very important in malignant growth, and most tumor cells have. a high level of insulin-like growth factor-receptors. The cross-reactivity of insulin and insulin-like growth factors and their receptors is known. In particular, insulin promotes the protein synthesis mainly in tumor cells because it is the agonist of its own receptor but also it is cross-agonist of insulin-like growth factor-receptors highly expressed in most malignant cells as it was referred.

It is noted, however, that other pharmaceutical compounds may be suitable for use in the method of the present invention for inducing protein synthesis in tumor cells, and that such pharmaceutical compounds may be utilized either in combination with or as a substitute for insulin. Among the other pharmaceutical compounds known to exhibit insulin-like growth factors are, somatotrophin, estrogens, androgens, just to name a few, however, it is to be understood that any compound able to induce protein synthesis in tumor cells may be suitable for use in the method of the present invention.

In addition to inducing protein synthesis in a plurality of tumor cells of the patient, the present invention comprises generating chemical-induced gene mutations or epigenetic protein modifications in the plurality of tumor cells by treating the patient with DNA targeted chemotherapeuticals, thereby resulting in the generation of a plurality of proteins unrecognizable as self-proteins by the patient's immune system which, as previously indicated, are known as neo-antigens or tumor associated antigens (TAA).

Most of the compounds used in antitumoral chemotherapy include agents structured to avoid DNA synthesis, which is required for cell reproduction. In particular, these compounds may comprise agents acting upon the structures of the DNA double helix that avoids the kinetic or enzymatic activity in DNA duplication, for example, cyclophosphamide, or enzymatic inhibitors acting upon enzymes required for nucleotide antecessor synthesis, such as, fluorouracil, or enzymes required for recovery of nucleotide synthesis cofactors including such compounds as methotrexate.

All compounds used in antitumoral chemotherapy which interfere with the normal DNA sequence can induce punctual or sectorial mutations through the modification of polypeptide codification. The significance of these mutations is that the immunologic non self-recognizance by the patient's immune system is higher when further mutagenic events are induced. In the present invention, at least one, but preferably a plurality of such mutagenic drugs, or DNA targeted chemotherapeuticals, may be utilized which are addressed with selectivity to the tumor cells. The selectivity of tumor cells is determined by the high level of expressed insulin-like growth factor-receptors, thereby allowing the DNA targeted chemotherapeuticals to reach the malignant cells through the increased permeability and proliferative requirements induced in these cells by the insulin.

In one alternate embodiment of the present invention, the method comprises promoting mutations in tumor cells via pharmacological agents and/or radiotherapeutical agents to produce chemical-induced or physical-induced gene mutations or epigenetic protein modifications, either in combination with or as a substitute for the aforementioned DNA targeted chemotherapeuticals.

At least one embodiment of the method of the present invention further comprises at least temporarily preserving the plurality of TAA within the plurality of cells of the patient. In one preferred embodiment, the plurality of TAA is at least temporarily preserved in the plurality of malignant tumor cells of the patient, by promoting the synthesis of molecules which act as chaperones of such intracellular peptides and proteins. The method of the present invention thus further comprises the step of inducing the synthesis of stress shock protein (SSP). The SSP is known as a chaperone because it protects proteins, such as TAA, by generating molecular complexes with them, thereby masking their presence to the immune system of the patient, as well as other molecular aggressors such as proteases. The induction of SSP may be accomplished utilizing pharmacological agents which are similar, and in at least one embodiment, identical to those utilized for generating the plurality of TAA. Thus, in at least one embodiment, the method of the present invention may accomplish the dual objectives, generating TAA and inducing SSP, in a single step. This is accomplished by the fact that the mechanisms involved in TAA generation, share the property of inducing SSP synthesis. Specifically, the present invention may employ the dual mechanisms of insulin hypoglycemia and chemotherapeutical induced stress.

More in particular, cells which are submitted to heat or other stress agents respond with the synthesis of a compound known as a heat shock protein (HSP) or, more generally, stress shock protein (SSP). The HSP or SSP have an inherent protective property for other cellular proteins or peptides by forming molecular complexes with them, at the risk, however, of the cellular proteins or peptides being denatured by the HSP or SSP. As the HSP or SSP form molecular complexes with these cellular proteins or peptides, the HSP and SSP are also commonly known as chaperones molecules.

In the method of the present invention, the plurality of tumor cells of the patient are exposed to such cellular stress via hypoglycemia and antitumoral chemotherapeuticals. As indicated above, this exposure is performed simultaneously with the generation of the plurality of TAA and, therefore, the chaperone molecules induced by the method preserve and at least temporarily store the plurality of TAA inside the plurality of tumor cells. In at least one embodiment, the method may also comprise administering indomethacin, cortisol derivatives, corticoid compounds, and other pharmacological agents to the patient to initiate the generation of SSP.

To elaborate further, when tumor cell stress is induced by hypoglycemia through insulin treatment, it is noted that insulin, which may also utilized by the method of the present invention for inducing protein synthesis, in sufficient dosages produces hypoglycemia, which induces SSP synthesis in cells subjected to this glucose restrictive condition. Because malignant cells normally require an elevated level of glycolysis to begin with, hypoglycemia presents a particularly high level of risk for these cells and, therefore, a particularly high level of stress, with a subsequent high level of SSP or chaperone molecule synthesis which may be utilized to at least temporarily preserve and store the plurality of TAA in the plurality of malignant tumor cells.

In one further embodiment, the method of the present invention may utilize other pharmacological or nutritional treatments to compliment the insulin induced hypoglycemia, or as a substitute for insulin to induce this condition in the patient so as to stress the plurality of tumor cells, thereby accomplishing the objective of generating SSP or chaperone molecules to at least temporarily preserve and store the plurality of TAA in the tumor cells.

In one alternate embodiment, stress to the tumor cells may be chemically induced by DNA targeted chemotherapeuticals. In particular, DNA targeted chemotherapeuticals, similar to those described above for use in mutagenic TAA generation, are also known for inducing cell stress. Active metabolites of cyclophosphamide, 5-fluorouracil, and methotrexate, are just a few of the drugs used in antitumoral chemotherapy which may also be employed by the present invention to chemically stress the patient's tumor cells to induce generation of SSP or chaperone molecules. As previously indicated, the method of the present invention may employ these drugs for simultaneously generating TAA and SSP.

The above method for generating SSP may be optimized when conducted in conjunction with indomethacin, a drug which is very well known for other uses in medicine, and has been recognized as a promoter of SSP synthesis. Indomethacin is a positive modulator of DNA binding to heat shock translational factor (hstf-1). This factor, through DNA binding, starts and maintains SSP synthesis.

In one other alternate embodiment, the method of the present invention may utilize other pharmacological or radiotherapeutical agents to complement or as substitutes for the DNA targeted chemotherapeuticals described above to chemically or physically stress tumor cells in the patient. Further, and as indicated above, indomethacin, cortisol derivatives, corticoid compounds, as well as other suitable pharmacologicals may be utilized in order to initiate SSP generation, thereby, enhancing the preserving and storing of the plurality of TAA in the plurality of tumor cells in the patient.

The method of the present invention further comprises the step of increasing the efficiency of the antitumoral immune response in cancer patients. More in particular, the presentation of an antigen to the immune system is facilitated by specific antigen presenting cells (APC), mainly to the lymphocytes, such presentation being necessary to elicit an immune response. At the same time, however, the antitumoral efficiency of this response requires avoiding the eliciting of an immune tolerance response to the plurality of TAA.

To begin, activating a plurality of APC may be accomplished via an adequate cytokine treatment, such as by administering a granulocyte-macrophage colony stimulating factor (GM-CSF). Human recombinant GM-CSF is known as an immune modulating cytokine that increases the dendritic cell population promoting its maturation and, as consequence, it amplifies the dendritic cell function of antigen presentation in order to start the immune response. This pharmacological property has been used to potentiate cancer vaccines with different external immunogens. In the present invention, and in particular, in an internal vaccine as previously described, the GM-CSF activated plurality of APC encounter the plurality of TAA which was previously preserved and stored in the plurality of tumor cells of the patient's body, which have been subsequently released into the patient's bloodstream via the mechanisms of autoschizis and/or apoptosis, which are described in further detail below. Additionally, the GM-CSF activated plurality of APC may encounter the plurality of TAA contained in an external vaccine comprising an autologous hemoderivative composition, as is also discussed in greater detail below.

In one embodiment, other pharmacological or immunological agents or biological response modifiers may be utilized to further increase the antitumoral immune response of GM-CSF, either as a complementary or substitutive methodological step.

In addition to increasing the encounters between the plurality of APC and the plurality of TAA, the method of the present invention further comprises breaking or inhibiting the immune tolerance response via pharmacological treatment and, in one preferred embodiment, by administering cyclophosphamide to the patient in a specific chronological sequence with the generation of the plurality of TAA.

Because the inventive method may be employed a plurality of times over the course of the patient's entire treatment regimen, it is necessary to minimize the immune tolerance response in the patient typically elicited by the immune-stimulation that has been described in cancer patients. Thus, the method of the present invention utilizes low dosages of cyclophosphamide in a specific chronological sequence with the antigenic stimulation to inhibit the immune tolerance response in the patient, prior to administration of both the internal vaccine and the external vaccine. It is to be understood that while the method of the present invention may utilize cyclophosphamide, it is not the exclusive means for breaking or inhibiting the immune tolerance response in the patient.

As indicated above, the present invention further comprises an internal vaccine. More specifically, the method comprises triggering the release of the plurality of TAA, which has been preserved and stored in the plurality of tumor cells of the patient, via a pharmacological tumor cell death that preserves the immunogenicity of the plurality of TAA, or immunogenic cell death.

It is known that all chemotherapeutical treatments in oncology kill tumor cells by apoptosis, but the immunogenicity of such tumor cells is only preserved if these tumor cells are first exposed to a cellular stress prior to being killed. Therefore, although known antitumoral chemotherapy comprise a mechanism to induce tumor cell death, a preferred embodiment of the present invention comprises a mechanism for inducing pharmacological tumor cell death utilizing a mechanism of cellular body fragmentation via high dosages of ascorbic acid administered intravenously, which is known as autoschizis, which may or may not be potentiated with menadione administered to the patient simultaneously with the high dosages of ascorbic acid. Thus, the method of the present invention triggers the release of the plurality of TAA, preserved and stored in the plurality of tumor cells of the patient, into the patient's body via the various intracellular components through the interstitial space including, but not limited to phagocytes, lymphatic vessels and/or blood vessels, thereby allowing the plurality of TAA to encounter the plurality of APC, and the patient's immune system, thus initiating an autologous antitumoral immune response.

Following some controversy in the medical field with regard to the effects of ascorbic acid on terminal cancer patients, it has been demonstrated that ascorbic acid in high doses administered intravenously is selectively cytotoxic for malignant cells and in high doses in vitro, it induces tumor cell death through a modified apoptosis mechanism known as autoschizis, as indicated above. Specifically, autoschizis is a cell death with fragmentation of the cell body and release of cell fragments and, more importantly with respect to the method of the present invention, the cell contents (i.e. the plurality of TAA) to the extracellular surroundings. As previously noted, other mechanisms of cell death, such as classical apoptosis, are only immunogenic if the cell has been exposed to the cellular stress prior to death, such that the antigens inside the cell are protected by chaperone compounds (e.g. SSP) induced by said stress, otherwise, the antigen may also be destroyed in the cell death process. The method of the present invention may comprise, in addition to the mechanism of cell death via autoschizis, a mechanism of cell death via chemotherapy-induced apoptosis, however, the apoptosis cell deaths will be immunogenic, because all of the tumor cells are exposed to cellular stress prior to death under the method of the present invention.

Currently, research is being conducted to determine the chemotherapeutic value of ascorbic acid in high dose administered intravenously to cancer patients, including the use of ascorbic acid potentiated by menadione. It is noted, however, that there are no known applications of ascorbic acid autoschizis, or any associated procedure, utilized to induce an antitumoral immune response or to start an immunotherapy treatment regimen. In the method of the present invention, an internal vaccine is obtained by triggering the release of the plurality of TAA from the plurality of malignant cells of the patient, at least partially into the patient's bloodstream via autoschizis. As such, specimens of blood containing at least some of the released plurality of TAA may subsequently be utilized to prepare an external vaccine, as discussed below.

At least one alternate embodiment of the method of the present invention further comprises the use of menadione or another pharmacological agent to potentiate the mechanism of autoschizis and/or the use of chemotherapy and/or the use of radiotherapy in combination with or as a substitute for the intravenous administration of ascorbic acid to induce tumor cell death and the subsequent, immunogenic release of the plurality of TAA into the patient's system.

The method of the present invention further comprises administering an external vaccine to cancer patients, the external vaccine preferably comprising an autologous hemoderivative composition prepared from an autologous blood specimen containing at least some of the plurality of TAA released from tumor cells.

The present invention further provides a method for producing such an external vaccine. In particular, at least some of the plurality of TAA released into the patient's blood as molecular chaperone protected complexes as a result of the internal vaccine are distributed in blood cells and blood plasma where they may become associated by external adhesion or by phagocytosis. When such a blood specimen is exposed to a hypotonic and hypothermic shock, essentially all of the plurality of TAA-chaperone complexes are released from the blood cells, into a supernatant. Afterwards, the supernatant may be exposed to thermal fractioning, such as by heating to approximately 100 degrees centigrade for approximately between 8 to 10 minutes. Under these conditions, the TAA-chaperone complexes are opened, and the plurality of TAA become free. In addition, under these conditions, a majority of the enzymatic and toxic properties of other molecules contained in the preparation are destroyed, but the immunogenic properties of the plurality of TAA is preserved. The method of the present invention further provides for filtration of the subsequent solution thereby resulting in the external vaccine comprising an autologous hemoderivative composition. The method of the present invention further provides for the inoculation of the patient, such as via subcutaneous injection, which is known for its efficiency to promote encounters between antigens and APC in other vaccination procedures.

A particular and significant advantage of preparing the external vaccine utilizing the method of the present invention is that the entire method requires minimal laboratory facilities, thereby providing a simple, safe, and economical method to prepare a vaccine, relative to those prepared in highly complex facilities where autologous biological specimens must be transported.

A more detailed description of the method for preparing an external vaccine comprising an autologous hemoderivative composition is as follows. The method of the present invention provides for extracting a blood specimen of approximately 20 milliliters from a femoral artery of the patient into a first syringe pre-filled with approximately 5,000 international units (I.U.) of heparin having a concentration in a range of between approximately 250 to 300 I.U. per milliliter. The blood specimen solution is allowed to sediment or settle in vertical position at a temperature of approximately 37 degrees centigrade. After approximately one hour, an aliquot of a supernatant of white cell rich blood plasma is separated from the blood specimen solution into a second syringe containing between approximately 3 to 4 parts of distilled water per part of the plasma-cell layer forming a plasma-cell solution and, thereby, inducing a hypotonic cytolysis. The method of the present invention further provides that the plasma-cell solution be stored at approximately minus twenty degrees centigrade for a period of approximately 24 hours, after which, the plasma-cell solution is warmed up to approximately 37 degrees centigrade in order to complete the hypotonic-hypothermic cytolysis process.

The resultant plasma-cell solution may be filtered through a glass wood membrane or optionally it is centrifuged at 2000 G, in order to clear the solution and remove gross precipitates. In yet another embodiment, the resultant plasma-cell solution may be sonicated to clear the solution and remove gross precipitates. The resultant plasma-cell solution is then subjected to further thermal treatment. More in particular, the method of the present invention permits utilization of any one of a plurality of thermal treatments in order to obtain different immunogens. In one preferred embodiment, the plasma-cell solution is heated to approximately 100 degrees centigrade for approximately between 8 to 10 minutes. The method also provides for allowing the solution to return to room temperature, approximately 25 degrees centigrade, until temperature equilibrium is reached. Finally, the resultant plasma-cell fraction may also be either filtered through glass wool membrane, centrifuged at 2000 G, or sonicated, followed by filtration through cellulose membranes ranging from between approximately 0.20 to 0.45 Am diameter.

While the method of the present invention for preparing an external vaccine presented above comprises one preferred embodiment, it is understood that alternative embodiments may be utilized to prepare an external vaccine comprising an autologous hemoderivative composition through modification of the methods for blood extraction, sedimentation, and/or specific temperatures and durations for thermal fractionation. In addition, it is understood that while a preferred embodiment of the present invention comprises subcutaneous inoculation of the patient with the external vaccine, inoculation via other mechanisms including, by way of example only, intradermal, intravenous, and/or intramuscular vaccination are encompassed in the method of the present invention to elicit an efficient antitumoral immune response or an antitumoral biological response targeted to tumor cells, tumor stroma patient's immune system and/or molecular mediators of the host biological response against cancer disease.

Thus, from the foregoing, it is readily seen that the method of the present invention allows one to elicit an antitumoral immune response in a cancer patient which may be addressed against his or her own specific tumor. In addition, the method provides for the pharmacological management of a patient's own cancer cell's antigen library to increase a malignant tumor's antigenicity. Also, the method comprises releasing an internal autologous vaccine from a patient's own tumor(s), specific antigens eliciting an antitumoral immune response against the patient's remaining malignant cancer cells. A further aspect of the present invention is a method of preparing and providing an external autologous vaccine comprising a hemoderivative composition obtained at least in part from inducing the generation and subsequent release into a patient's bloodstream of tumor specific TAA. Yet another aspect of the present invention is a method to enhance the antitumoral immune response in a cancer patient by activating an APC population induced by cytokine treatment and inhibiting tolerance immune response in the patient. The present invention further provides a method for an immunologically assessing an immune response elicited by an autologous vaccine of a cancer patient via an intradermal test, as well as a method for assessing an antitumoral response elicited by an autologous vaccine of the cancer patient. Most importantly, the method of the present invention provides an innovative and alternate technique for eliciting an antitumoral immune response in a cancer patient in the event that surgery, chemotherapy, radiotherapy, and/or other cancer treatment regimens fail.

These and other objects, features and advantages of the present invention will become more clear when the figures as well as the detailed description are taken into consideration.

### BRIEF DESCRIPTION OF THE DRAWINGS

For a fuller understanding of the nature of the present invention, reference should be had to the following detailed description taken in connection with the accompanying figures in which:

FIG. 1 is a schematic view of one preferred embodiment of the inventive method to elicit an effective autologous antitumoral immune response in a patient.

FIG. 2 is a schematic of the embodiment of FIG. 1 further illustrating one preferred embodiment for generating and preserving a plurality of tumor associated antigens (TAA) in a plurality of cells in the patient.

FIG. 3 is a schematic of the embodiment of FIG. 1 further illustrating one preferred embodiment for activating a plurality of antigen presenting cells (APC) in the patient.

FIG. 4 is a schematic of the embodiment of FIG. 1 further illustrating one preferred embodiment for inhibiting an immune tolerance response for the TAA in the patient.

FIG. 5 is a schematic of the embodiment of FIG. 1 further illustrating one preferred embodiment for triggering an internal vaccine in the patient.

FIG. 6 is a schematic of the embodiment of FIG. 1 further illustrating one preferred embodiment for preparing and providing an external vaccine to the patient.

### DETAILED DESCRIPTION OF THE PREFERRED EMBODIMENT

While this invention is susceptible of embodiment in many different forms, there is shown in the figures and will herein be described in detail at least one specific embodiment, with the understanding that the present disclosure is to be considered as an exemplification of the principles of the invention and is not intended to limit the invention to the embodiment illustrated.

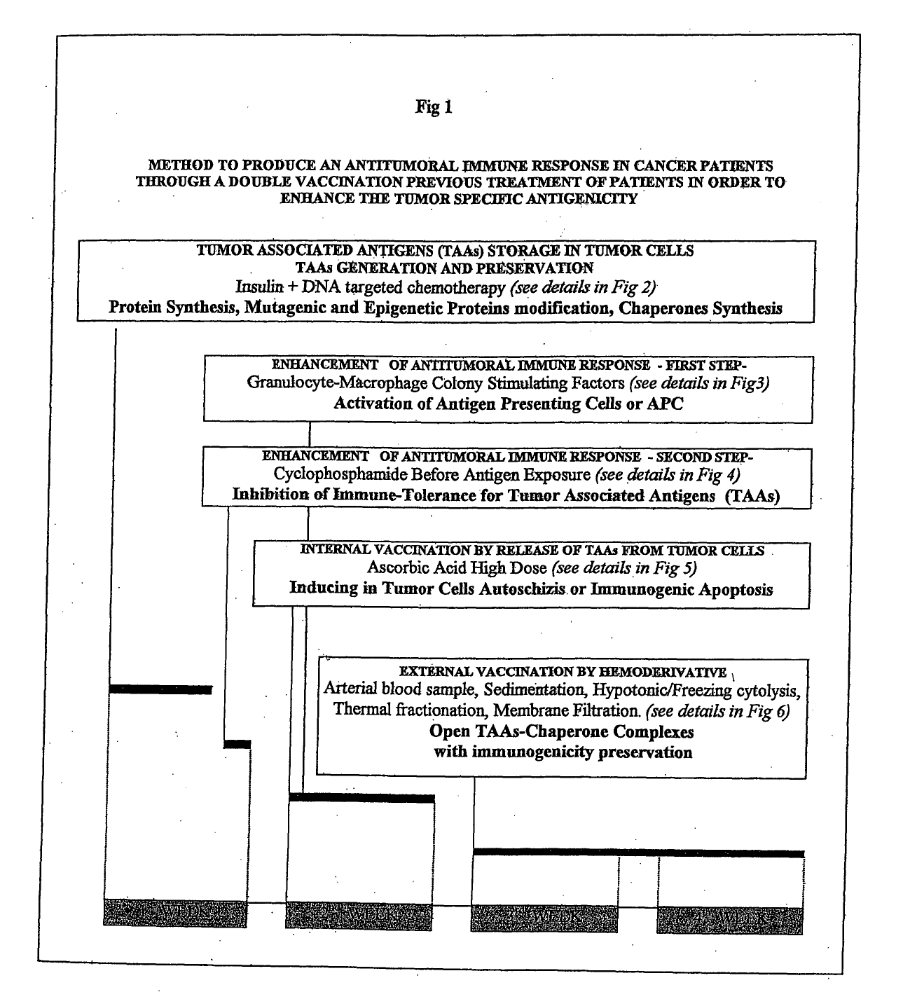

As indicated above, the present invention is directed in general to a new, improved, and innovative active specific immunotherapy technique. More in particular, the present invention is directed to a method and a composition to elicit an effective antitumoral immune response in a patient, specific to his or her own tumor antigens (i.e. an autologous antitumoral immune response). Thus, more specifically, the present invention is directed to a method and composition to elicit an effective autologous antitumoral immune response in a cancer patient which comprises generating, preserving, and storing specific tumor associated antigens, and eliciting the autologous antitumoral immune response, at least in part, through a combination of dual vaccines. In addition, the present invention provides enhancement of the antitumoral immune response resulting from an internal vaccine and an external vaccine by activating antigen presenting cells, as well as by inhibiting a tolerance immune response in cancer patients. The present invention further provides a method for preparing a hemoderivative composition, and, more in particular, an autologous hemoderivative composition, for utilization in the inventive method as an external vaccine. FIG. 1 presents a schematic view of one preferred embodiment of the method of the present invention. More in particular, FIG. 1 illustrates one preferred embodiment of one complete treatment cycle of the method of the present invention. The method of the present invention may comprise completing a single treatment cycle, however, at least one embodiment of the present invention includes completing a plurality of treatment cycles.

To begin, the method of the present invention provides for generating a plurality of tumor associated antigens (TAA) in a plurality of cells of the patient. As indicated above, mutated genes can generate a plurality of TAA by their direct expression or by the promotion of intracellular conditions eliciting epigenetic normal protein transformation. In order to generate the production of a plurality of TAA in tumor cells, it is necessary to increase in these cells their protein synthesis and mutation frequency.

Thus, at least one embodiment of the method of the present invention further comprises inducing protein synthesis in a plurality of tumor cells by treating the patient with a suitable pharmaceutical compound in order to activate the growth factor-receptors, such as are typically highly expressed in most malignant cells. One pharmaceutical compound which is suitable for this purpose is insulin, due to the insulin-like growth factor-receptors which are highly expressed in many malignant cells. In particular, insulin promotes the protein synthesis mainly in tumor cells because it is the agonist of its own receptor but also it is cross-agonist of insulin-like growth factor-receptors highly expressed in most malignant cells as it was referred.

It is noted, however, that other pharmaceutical compounds may be suitable for use in the method of the present invention for inducing protein synthesis in tumor cells, and that such pharmaceutical compounds may be utilized either in combination with or as a substitute for insulin. Among the other pharmaceutical compounds known to exhibit insulin-like growth factors are, somatotrophin, estrogens, androgens, just to name a few, however, it is to be understood that any compound able to induce protein synthesis in tumor cells may be suitable for use in an embodiment of the method of the present invention.

In addition to inducing protein synthesis in the plurality of cells of the patient, the present invention comprises generating chemical-induced gene mutations or epigenetic protein modifications in the plurality of tumor cells by treating the patient with DNA targeted chemotherapeuticals, thereby resulting in the generation of a plurality of proteins unrecognizable as self-proteins by the patient's immune system which, as previously indicated, are known as neo-antigens or tumor associated antigens (TAA).

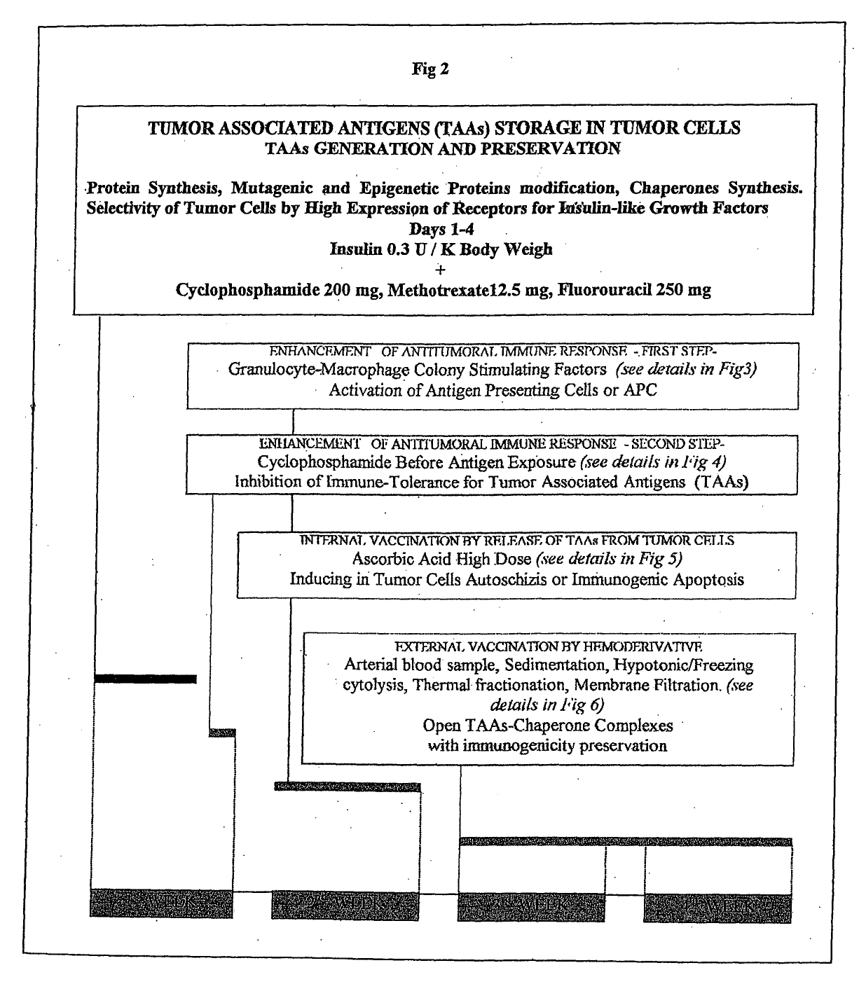

Many of the compounds used in antitumoral chemotherapy include agents structured to avoid DNA synthesis, which is required for cell reproduction. In particular, DNA targeted chemotherapeuticals comprise agents which act upon the structures of the DNA double helix that avoid the kinetic or enzymatic activity in DNA duplication and include, but are not limited to, cyclophosphamide, or enzymatic inhibitors acting upon enzymes required for nucleotide antecessor synthesis, such as, fluorouracil, or enzymes required for recovery of nucleotide synthesis cofactors including such compounds as methotrexate. One embodiment of the present invention may comprise administering at least one of these DNA targeted chemotherapeuticals to the patient during a preparatory treatment phase. In one preferred embodiment, as illustrated in FIG. 2, the method of the present invention comprises administering a plurality of DNA targeted chemotherapeuticals to the patient during the preparatory treatment phase.

Thus, the method of the present invention provides for administering one or more compounds to the patient during a preparatory phase of the treatment cycle for generating a plurality of TAA. The compounds selected to be administered to the patient are among those know to induce protein synthesis, such as insulin, as well as to generate production of a plurality of tumor associated antigens (TAA) in a plurality of the cells, and in one preferred embodiment, the malignant tumor cells of the patient.

As illustrated in FIG. 2, one preferred embodiment of the method of the present invention comprises administering insulin to the patient each day of the preparatory treatment phase or, more specifically, days one through four of the treatment cycle, at a daily dosage of approximately 0.3 international units (I.U.) per kilogram of body weight. In addition, FIG. 2 also illustrates that in a preferred embodiment, a plurality of DNA targeted chemotherapeuticals are administered to the patient during the preparatory treatment phase (e.g. days one through four of the treatment cycle). Specifically, one preferred embodiment of the method of the present invention comprises administering cyclophosphamide, at a daily dosage in a range of between approximately 100 to 200 milligrams, methotrexate, at a daily dosage in a range of between approximately 2.5 to 12.5 milligrams, and fluorouracil, at a daily dosage in a range of between approximately 125 to 250 milligrams, to the patient each day of the preparatory treatment phase. In at least one embodiment of the present invention, the preparatory phase comprises days one through five of the treatment cycle.

At least one embodiment of the method of the present invention further comprises at least temporarily preserving the plurality of TAA within the plurality of cells of the patient. In one preferred embodiment, the plurality of TAA is at least temporarily preserved in the plurality of malignant tumor cells of the patient, by promoting the synthesis of molecules which act as chaperones of such intracellular peptides and proteins. The method of the present invention thus further comprises the step of inducing the synthesis of stress shock protein (SSP). The SSP is known as a chaperone because it protects proteins, such as TAA, by generating molecular complexes with them, thereby masking their presence to the immune system of the patient, as well as other molecular aggressors such as proteases. The induction of SSP may be accomplished utilizing pharmacological agents which are similar, and in at least one embodiment, identical to those utilized for generating the plurality of TAA. Thus, in at least one embodiment, the method of the present invention may accomplish the dual objectives of generating TAA and inducing SSP in a single step. This is accomplished by the fact that the mechanisms involved in TAA generation, are similar to those for inducing the synthesis of SSP. Specifically, the present invention may employ the dual mechanisms of insulin hypoglycemia and chemotherapeutical induced stress.

Thus, in the method of the present invention, the plurality of tumor cells of the patient are exposed to cellular stress via hypoglycemia and antitumoral chemotherapeuticals. As indicated above, this exposure is performed simultaneously with the generation of the plurality of TAA and, therefore, the chaperone molecules induced by the method preserve and at least temporarily store the plurality of TAA inside the plurality of tumor cells. In at least one embodiment, the method may further comprise administering indomethacin, cortisol derivatives, corticoid compounds, and other pharmacological agents to the patient to initiate the generation of the plurality of SSP.

As such, at least one embodiment of the present invention further comprises preserving the plurality of TAA by inducing the synthesis of a plurality of SSP. More in particular, the method of the present invention may include inducing the synthesis of the SSP comprises by administering indomethacin to the patient. In one alternate embodiment, the method of the present invention may include inducing the synthesis of the SSP by administering a corticoid compound to the patient.

Also, as indicated above, the method of the present invention further comprises storing the TAA in the plurality of cells of the patient by inducing the synthesis of a plurality of stress shock proteins (SSP). Once again, the method may comprise inducing the synthesis of the SSP comprises by administering indomethacin to the patient. In one alternate embodiment, the method of the present invention may include inducing the synthesis of the SSP by administering a corticoid compound to the patient.

The method of the present invention also comprises breaking or inhibiting the immune tolerance response relative to the TAA generated in the cells of the patient to enhance the antitumoral immune response. In at least one embodiment, inhibiting the immune tolerance response is accomplished via pharmacological treatment and, in one preferred embodiment, by administering cyclophosphamide to the patient in a specific chronological sequence with the generation of the plurality of TAA.

Because the inventive method may comprise completing a plurality of treatment cycles over the course of the patient's entire treatment regimen, it becomes necessary to minimize the immune tolerance response in the patient typically elicited by the immune-stimulation that has been described in cancer patients. Thus, in at least one embodiment, the method of the present invention utilizes low dosages of cyclophosphamide to inhibit the immune tolerance response in the patient, prior to administration of both the internal vaccine and the external vaccine. It is to be understood that while the method of the present invention may utilize cyclophosphamide, it is not the exclusive means for breaking or inhibiting the immune tolerance response in the patient encompassed by and which may be utilized in conjunction with the method of the present invention.

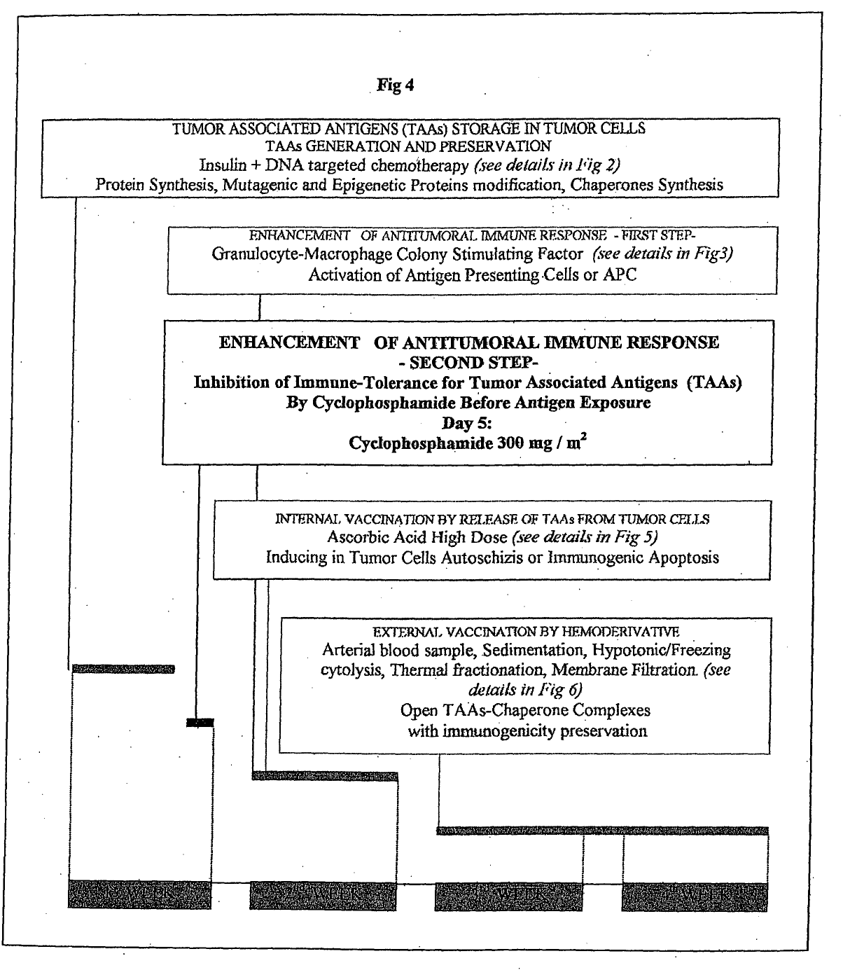

In particular, one embodiment of the method of the present invention comprises administering cyclophosphamide to the patient during a first intermediate phase of the treatment cycle. More specifically, and as illustrated in FIG. 4, one preferred embodiment of the method of the present invention comprises administering cyclophosphamide at a dosage of approximately 300 milligrams per square meter of surface area of the patient's body, on day five of the treatment cycle.

The method of the present invention further comprises activating a plurality of antigen presenting cells (APC) in the patient to further enhance the antitumoral immune response. More in particular, the presentation of an antigen to the immune system is facilitated by specific APC, mainly to the lymphocytes, and is necessary to elicit an immune response. At the same time, however, the antitumoral efficiency of this response requires avoiding the eliciting of an immune tolerance response to the plurality of TAA.

In at least one embodiment, activating a plurality of APC may be accomplished via an adequate cytokine treatment, such as, for example, administering a granulocyte-macrophage colony stimulating factor (GM-CSF). Human recombinant GM-CSF is known as an immune modulating cytokine that increases the dendritic cell population promoting its maturation and, as consequence, it amplifies the dendritic cell function of antigen presentation in order to start the immune response. In the present invention, and in particular, in conjunction with an internal vaccine as previously described, the GM-CSF activated plurality of APC encounter the plurality of TAA released into the patient's bloodstream via the mechanisms of autoschizis and/or apoptosis, as previously described in detail. Additionally, the GM-CSF activated plurality of APC may encounter the plurality of TAA contained in an external vaccine comprising an autologous hemoderivative composition, as also discussed in further detail below. It is understood to be within the scope of the method of the present invention to administer alternate pharmacological or immunological agents or biological response modifiers to either increase the antitumoral immune response of GM-CSF, or as a substitute for GM-CSF.

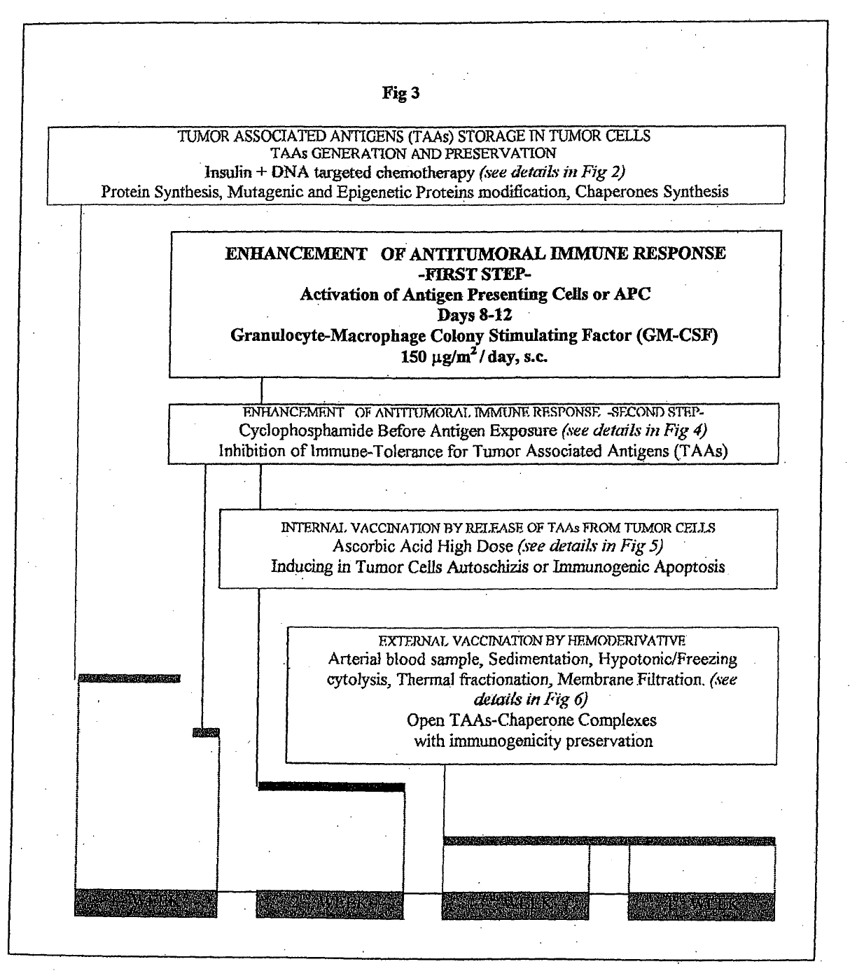

In one preferred embodiment, the method of the present invention comprises activating a plurality of antigen presenting cells (APC), to further enhance the antitumoral immune response in the patient, by administering a cytokine to the patient during a primary treatment phase. In at least one embodiment, the method of the present invention includes administering the cytokine comprising granulocyte-macrophage colony stimulating factor (GMCSF) to the patient. As illustrated in FIG. 3, one preferred embodiment of the method of the present invention comprises administering GM-CSF to the patient on each day of the primary treatment phase at a daily dosage in a range of between approximately 150 to 250 micrograms. In at least one embodiment, the primary treatment phase of the method comprises day eight through twelve of the treatment cycle.

As discussed above in some detail, the method of the present invention further comprises the new and innovative feature of triggering an internal vaccine in the patient. As disclosed herein, the internal vaccine comprises the release of the TAA previously generated, preserved, and stored in the plurality of tumor cells of the patient's body, via a tumor cell death that preserves the immunogenicity of the TAA, known as an immunogenic cell death.

In particular, the method of the present invention provides for triggering the release of the plurality of TAA into the patient's body via the various intracellular components through the interstitial space including, but not limited to phagocytes, lymphatic vessels and/or blood vessels, thereby allowing the plurality of TAA to encounter the plurality of APC, and the patient's immune system, thereby initiating an autologous antitumoral immune response. In at least one embodiment, the method of the present invention includes administering ascorbic acid to the patient during the primary treatment phase to induce immunogenic cell death through a modified apoptosis mechanism known as autoschizis. In one preferred embodiment, the internal vaccine is triggered via administering the ascorbic acid to the patient intravenously, for example, in a lactate-ringer solution.

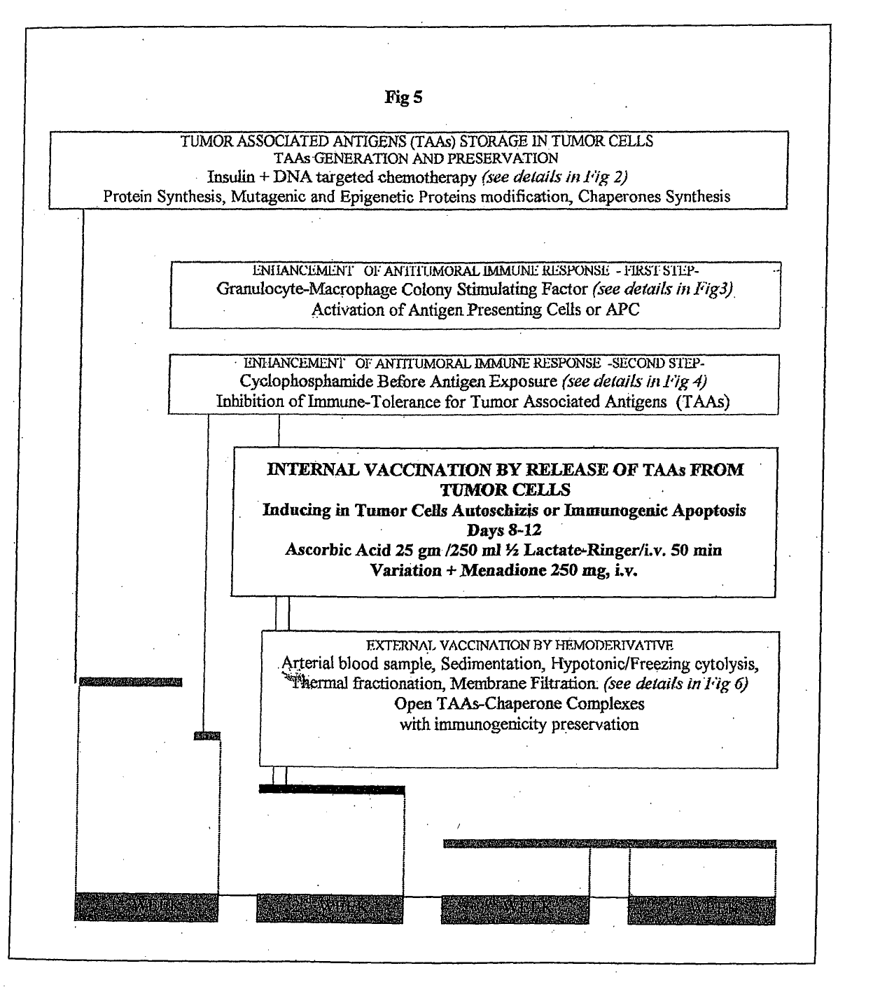

As illustrated in FIG. 5, a preferred embodiment of the present invention comprises triggering the internal vaccine by inducing autoschizis by administering ascorbic acid to the patient during each day of the primary treatment phase. More specifically, the preferred embodiment of the method includes administering the ascorbic acid to the patient each day of the primary treatment phase at a daily dosage of approximately 25 grams in approximately 250 milliliters of a lactate-ringer solution. As noted above, the ascorbic acid is preferable administered intravenously. As also noted above, in at least one embodiment of the method of the present invention, the primary treatment phase includes days eight through twelve of the treatment cycle.

Alternatively, the method may comprise, either in lieu of or in addition to the mechanism of cell death via autoschizis, a mechanism of cell death via chemotherapy-induced apoptosis, however, the apoptosis cell death induced by the method of the present invention will be an immunogenic cell death, because all of the tumor cells are exposed to cellular stress prior to death.

At least one embodiment of the method of the present invention further comprises the administration of menadione or another pharmacological agent to potentiate the mechanism of autoschizis and/or the use of chemotherapy and/or the use of radiotherapy in combination with or as a substitute for the intravenous administration of ascorbic acid to induce the immunogenic cell death and the subsequent, immunogenic release of the plurality of TAA into the patient's system.

As FIG. 5 further illustrates, an alternate embodiment of the method of the present invention further comprises administering the menadione to the patient during the primary treatment phase (e.g. days eight through twelve of the treatment cycle). Specifically, the method of the present invention includes administering the menadione to the patient each day of the primary treatment phase at a daily dosage of approximately 250 milligrams. In one preferred embodiment, the method comprises administering the menadione to the patient intravenously, however, in at least one alternate embodiment, the menadione may be administered orally.

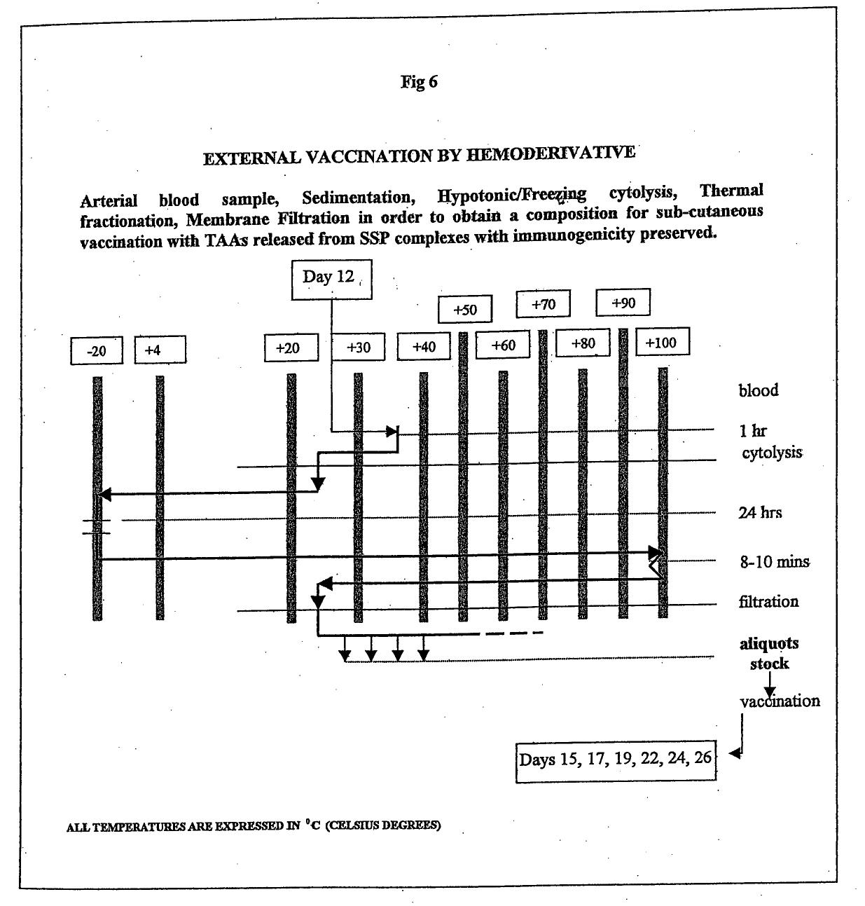

The method of the present invention further comprises providing an external vaccine to cancer patients, the external vaccine comprising a hemoderivative composition, and preferably, an autologous hemoderivative composition prepared from a blood specimen from the patient and, thus, containing at least some of the plurality of TAA released from tumor cells. In particular, the method of the present invention includes administering an external vaccine to the patient during a secondary phase of the treatment cycle. As illustrated in FIG. 6, in one preferred embodiment, the external vaccine is administered to the patient on each of days fifteen, seventeen, nineteen, twenty-two, twenty-four, and twenty-six of the treatment cycle. It is well understood that numerous variations of this preferred schedule for administering the external vaccine during the secondary treatment phase are encompassed by the scope of the method of the present invention.

In at least one embodiment, the external vaccine is administered subcutaneously, however, it is also understood to be within the scope of the present invention to include administering the external vaccine to the patient via alternate inoculation mechanisms, including, but not limited to, intradermal and intramuscular inoculations.

One alternate embodiment of the present invention further comprises administering cyclophosphamide to the patient each day of a second intermediate treatment phase at a daily dosage of approximately 300 milligrams per square meter of surface area of the patient's body. In at least one embodiment, the second intermediate treatment phase comprises day thirteen of the treatment cycle.

The present invention further comprises a method for preparing the autologous hemoderivative composition, as illustrated schematically in FIG. 6, for use in eliciting an effective antitumoral immune response in a patient, such as may be utilized in the method described herein. In one preferred embodiment, the method for preparing the autologous hemoderivative composition includes extracting a blood specimen of approximately 20 milliliters from a femoral artery of the patient into a first syringe pre-filled with approximately 5,000 international units (I.U.) of heparin having a concentration in a range of between approximately 250 to 300 I.U. per milliliter. The blood specimen solution is allowed to settle while maintained in vertical position at a temperature of approximately 37 degrees centigrade. After approximately one hour, an aliquot of a supernatant of white cell rich blood plasma is separated from the blood specimen solution into a second syringe containing between approximately 3 to 4 parts of distilled water per part of the plasma-cell layer thereby forming a plasma-cell solution and, inducing a hypotonic cytolysis. The method of the present invention further provides that the plasma-cell solution be stored at approximately minus twenty degrees centigrade for a period of approximately 24 hours, after which, the plasma-cell solution is warmed up to approximately. 37 degrees centigrade in order to complete a hypotonic-hypothermic cytolysis process.

The resultant plasma-cell solution may be filtered through a glass wood membrane or optionally it may be centrifuged at 2000 G, in order to clear the solution and remove gross precipitates. In yet another embodiment, the resultant plasma-cell solution may be sonicated to clear the solution and remove the precipitates. The resultant plasma-cell solution is then subjected to further thermal treatment. More in particular, the method of the present invention permits utilization of any one of a plurality of thermal treatments in order to obtain different immunogens. In one preferred embodiment, the plasma-cell solution is heated to approximately 100 degrees centigrade for approximately between 8 to 10 minutes, thereby forming a plasma-cell fraction. The method also provides for allowing the solution to return to room temperature, approximately 25 degrees centigrade, until temperature equilibrium is reached. Finally, the resultant plasma-cell fraction may also be either filtered through glass wool membrane, centrifuged at 2000 G, or sonicated, followed by filtration through cellulose membranes ranging from between approximately 0.20 to 0.45 μm diameter.

While the method of the present invention for preparing an external vaccine presented above comprises one preferred embodiment, it is understood that alternative embodiments may be utilized to prepare an external vaccine comprising an autologous hemoderivative composition through modification of the methods for blood extraction, sedimentation, and/or specific temperatures and durations for thermal fractionation. In addition, it is understood that while a preferred embodiment of the present invention comprises subcutaneous inoculation of the patient with the external vaccine, inoculation via other mechanisms including, by way of example only, intradermal, intravenous, and/or intramuscular inoculation are encompassed in the method of the present invention to elicit an efficient antitumoral immune response or an antitumoral biological response targeted to tumor cells, tumor stroma patient's immune system and/or molecular mediators of the host biological response against cancer disease.

The present invention is also directed towards an autologous hemoderivative composition, which may be utilized as an external vaccine in the method to elicit an effective antitumoral immune response disclosed herein. In one preferred embodiment, the autologous hemoderivative composition comprises a plasma-cell solution which has been cooled to approximately minus twenty degrees centigrade for approximately 24 hours. The cooled plasma-cell solution may subsequently be heated to approximately 100 degrees centigrade and fractioned for between approximately 8 to 10 minutes thereby forming a plasma-cell fraction, after which, the plasma-cell fraction may be filtered upon cooling, and readied to provide to the patient as an external vaccine.

The plasma-cell solution of the autologous hemoderivative composition may be at least partially defined by a supernatant plasma-cell layer which is separated from a blood specimen solution and a quantity of distilled water, typically, 3 to 4 parts of distilled water per part of the blood specimen solution.

In addition, the blood specimen solution may comprise a blood specimen extracted from a patient, preferably, from femoral artery of the patient, into a solution comprising, in at least one embodiment, approximately 5,000 international units (I.U.) of heparin at a concentration in a range of between approximately 250 to 300 I.U. per milliliter.

Since many modifications, variations and changes in detail can be made to the described preferred embodiment of the invention, it is intended that all matters in the foregoing description and illustrated in the accompanying figures be interpreted as illustrative and not in a limiting sense. Thus, the scope of the invention should be determined by the appended claims and their legal equivalents.

Now that the invention has been described, what is claimed is:

1. A method to elicit an effective antitumoral immune response in a patient comprising:
generating a plurality of tumor associated antigens (TAA) in a plurality of cells in the patient,
inhibiting an immune tolerance response relative to the TAA in the patient to enhance the antitumoral immune response,
activating a plurality of antigen presenting cells (APC) in the patient to further enhance the antitumoral immune response,
triggering an internal vaccine in the patient to at least partially elicit the antitumoral immune response, and
providing an external vaccine to the patient to further elicit the antitumoral immune response.

2. A method as recited in claim 1 further comprising generating the TAA in a plurality of tumor cells of the patient.

3. A method as recited in claim 1 further comprising preserving the TAA in the patient.

4. A method as recited in claim 3 wherein preserving the TAA further comprises inducing the synthesis of a plurality of stress shock proteins (SSP).

5. A method as recited in claim 4 wherein inducing the synthesis of the SSP comprises administering indomethacin to the patient.

6. A method as recited in claim 4 wherein inducing the synthesis of the SSP comprises administering a corticoid compound to the patient.

7. A method as recited in claim 1 further comprising storing the TAA in the plurality of cells of the patient.

8. A method as recited in claim 7 wherein storing the TAA further comprises inducing the synthesis of a plurality of stress shock proteins (SSP).

9. A method as recited in claim 8 wherein inducing the synthesis of the SSP comprises administering indomethacin to the patient.

10. A method as recited in claim 8 wherein inducing the synthesis of the SSP comprises administering a corticoid compound to the patient.

11. A method as recited in claim 1 wherein generating the TAA further comprises inducing protein synthesis in the plurality of cells of the patient.

12. A method as recited in claim 1 further comprising inducing protein synthesis in the plurality of cells of the patient via administering a pharmaceutical compound to the patient.

13. A method as recited in claim 12 further comprising administering a pharmaceutical compound to the patient comprising insulin.

14. A method as recited in claim 1 wherein generating the TAA further comprises administering insulin to the patient.

15. A method as recited in claim 1 wherein generating the TAA comprises administering at least one DNA targeted chemotherapeutical to the patient.

16. A method as recited in claim 15 further comprising administering at least one DNA targeted chemotherapeutical comprising cyclophosphamide to the patient.

17. A method as recited in claim 15 further comprising administering at least one DNA targeted chemotherapeutical comprising methotrexate to the patient.

18. A method as recited in claim 15 further comprising administering at least one DNA targeted chemotherapeutical comprising fluorouracil to the patient.

19. A method as recited in claim 1 wherein activating the APC in the patient comprises administering a cytokine to the patient.

20. A method as recited in claim 19 further comprising administering the cytokine comprising granulocyte-macrophage colony stimulating factor (GM-CSF) to the patient.

21. A method as recited in claim 1 wherein inhibiting the immune tolerance response for the TAA comprises administering cyclophosphamide to the patient.

22. A method as recited in claim 1 wherein triggering the internal vaccine in the patient comprises inducing cell death in the plurality of cells in the patient.

23. A method as recited in claim 22 further comprising inducing immunogenic cell death in a plurality of tumor cells in the patient.

24. A method as recited in claim 22 further comprising inducing immunogenic cell death in the plurality of cells in the patient via apoptosis.

25. A method as recited in claim 24 further comprising exposing the plurality of cells to cellular stress prior to inducing immunogenic cell death in the plurality of cells in the patient via apoptosis.

26. A method as recited in claim 22 further comprising inducing immunogenic cell death in the plurality of cells in the patient via autoschizis.

27. A method as recited in claim 26 wherein inducing immunogenic cell death in the plurality of cells in the patient via autoschizis comprises administering ascorbic acid to the patient.

28. A method as recited in claim 27 further comprising administering the ascorbic acid to the patient intravenously.

29. A method as recited in claim 27 wherein inducing immunogenic cell death in the plurality of cells in the patient via autoschizis further comprises simultaneously administering menadione to the patient.

30. A method as recited in claim 29 further comprising administering menadione to the patient intravenously.

31. A method as recited in claim 1 wherein providing the external vaccine to the patient further comprises inoculating the patient with the external vaccine subcutaneously.

32. A method as recited in claim 1 wherein providing the external vaccine to the patient further comprises inoculating the patient with the external vaccine via intradermal inoculation.

33. A method as recited in claim 1 wherein providing the external vaccine to the patient further comprises inoculating the patient with the external vaccine via intramuscular inoculation.

34. A method to elicit an effective antitumoral immune response in a patient comprising:
completing at least one treatment cycle, each treatment cycle comprising,
administering insulin to the patient during a preparatory treatment phase,
administering at least one DNA targeted chemotherapeutical to the patient during the preparatory treatment phase,
administering cyclophosphamide to the patient during a first intermediate treatment phase,
administering a cytokine to the patient during a primary treatment phase,
administering ascorbic acid to the patient during the primary treatment phase,
administering menadione to the patient during the primary treatment phase, and
administering a hemoderivative composition to the patient during a secondary treatment phase.

35. A method as recited in claim 34 further comprising administering the insulin to the patient on each of days one through four of the treatment cycle.

36. A method as recited in claim 34 further comprising administering the insulin to the patient on each of days one through five of the treatment cycle.

37. A method as recited in claim 34 further comprising administering the insulin to the patient each day of the preparatory treatment phase at a daily dosage of approximately 0.3 international units per kilogram of the patient's body weight.

38. A method as recited in claim 34 further comprising administering the at least one DNA targeted chemotherapeutical to the patient on each of days one through four of the treatment cycle.

39. A method as recited in claim 34 further comprising administering the at least one DNA targeted chemotherapeutical to the patient on each of days one through five of the treatment cycle.

40. A method as recited in claim 34 further comprising administering the at least one DNA targeted chemotherapeutical comprising cyclophosphamide to the patient each day of the preparatory treatment phase at a daily dosage in a range of between approximately 100 to 200 milligrams.

41. A method as recited in claim 34 further comprising administering the at least one DNA targeted chemotherapeutical comprising methotrexate to the patient each day of the preparatory treatment phase at a daily dosage in a range of between approximately 2.5 to 12.5 milligrams.

42. A method as recited in claim 34 further comprising administering the at least one DNA targeted chemotherapeutical comprising fluorouracil to the patient each day of the preparatory treatment phase at a daily dosage in a range of between approximately 125 to 250 milligrams.

43. A method as recited in claim 34 further comprising administering the cyclophosphamide to the patient on day five of the treatment cycle.

44. A method as recited in claim 34 further comprising administering the cyclophosphamide to the patient each day of the first intermediate treatment phase at a daily dosage of approximately 300 milligrams per square meter of surface area of the patient's body.

45. A method as recited in claim 34 further comprising administering the cytokine to the patient on each of days eight through twelve of the treatment cycle.

46. A method as recited in claim 34 further comprising administering the cytokine comprising granulocyte-macrophage colony stimulating factor (GM-CSF) to the patient on each day of the primary treatment phase at a daily dosage in a range of between approximately 150 to 250 micrograms.

47. A method as recited in claim 34 further comprising administering the ascorbic acid to the patient on each of days eight through twelve of the treatment cycle.

48. A method as recited in claim 34 further comprising administering the ascorbic acid to the patient each day of the primary treatment phase at a daily dosage of approximately 25 grams in a solution of approximately 250 milliliters of a lactate-ringer solution.

49. A method as recited in claim 48 further comprising administering the ascorbic acid to the patient intravenously.

50. A method as recited in claim 34 further comprising administering the menadione to the patient on each of days eight through twelve of the treatment cycle.

51. A method as recited in claim 34 further comprising administering the menadione to the patient each of day the primary treatment phase at a daily dosage of approximately 250 milligrams.

52. A method as recited in claim 51 further comprising administering the menadione to the patient intravenously.

53. A method as recited in claim 51 further comprising administering menadione to the patient orally.

54. A method as recited in claim 34 further comprising administering the hemoderivative composition to the patient on each day of the secondary treatment phase.

55. A method as recited in claim 34 further comprising administering an autologous hemoderivative composition to the patient on each day of the secondary treatment phase.

56. A method as recited in claim 34 further comprising administering the hemoderivative composition to the patient on each of days fifteen, seventeen, nineteen, twenty-two, twenty-four, and twenty-six of the treatment cycle.

57. A method as recited in claim 34 further comprising administering cyclophosphamide to the patient each day of a second intermediate treatment phase at a daily dosage of approximately 300 milligrams per square meter of surface area of the patient's body.

58. A method as recited in claim 57 further comprising administering the cyclophosphamide to the patient on day thirteen of the treatment cycle.

59. A method as recited in claim 34 further comprising completing a plurality of treatment cycles.

60. A method of preparation of an autologous hemoderivative composition for use in eliciting an effective antitumoral immune response in a patient comprising:
extracting a blood specimen from the patient and forming a blood specimen solution,
separating a supernatant plasma-cell layer from the blood specimen solution after settling,
diluting the supernatant plasma-cell layer in a dilutant forming a plasma-cell solution and thereby inducing a hypotonic shock,
cooling and heating the plasma-cell solution and thereby inducing a hypothermic shock,
fractioning the plasma-cell solution by heating to a predetermined temperature for a predetermined period of time and forming a plasma-cell fraction, and filtering the plasma-cell fraction prior to administrating to the patient.

61. A method of preparation as recited in claim 60 further comprising extracting approximately 20 milliliters of the blood specimen from the femoral artery of the patient into a heparin solution thereby forming the blood specimen solution.

62. A method of preparation as recited in claim 60 further comprising settling the blood specimen solution for approximately one hour and separating the supernatant plasma-cell layer.

63. A method of preparation as recited in claim 60 further comprising diluting the supernatant plasma-cell layer in distilled water at a ratio in a range of approximately 3 to 4 parts distilled water per 1 part supernatant plasma-cell layer, thereby forming the plasma-cell solution.

64. A method of preparation as recited in claim 60 further comprising cooling the plasma-cell solution to approximately minus twenty degrees centigrade for approximately 24 hours.

65. A method of preparation as recited in claim 60 further comprising fractioning the plasma-cell solution by heating to approximately one hundred degrees centigrade for between approximately 8 to 10 minutes.

66. An autologous hemoderivative composition comprising:
a plasma-cell solution cooled to approximately minus twenty degrees centigrade for approximately 24 hours, and subsequently heated to approximately 100 degrees centigrade for between approximately 8 to 10 minutes, and filtered after cooling,
said plasma-cell solution being defined by a supernatant plasma-cell layer separated from a blood specimen solution and a quantity of distilled water, and
said blood specimen solution comprising a blood specimen extracted from a femoral artery of a patient and a heparin solution.
